(19) United States
(12) Patent Application Publication
(10) Pub. No.: US 2018/0161095 A1
(43) Pub. Date: Jun. 14, 2018

(54) BIOELECTRONIC DEVICES, SYSTEMS, AND USES THEREOF

(71) Applicant: Georgia Tech Research Corporation, Atlanta, GA (US)

(72) Inventor: HUA WANG, ATLANTA, GA (US)

(21) Appl. No.: 15/576,844

(22) PCT Filed: May 26, 2016
(86) PCT No.: PCT/US16/34255

§ 371 Date: Nov. 26, 2017

Related U.S. Application Data
(60) Provisional application No. 62/166,459, filed on May 26, 2015.

(51) Int. Cl. A61B 18/18; G01N 21/62; A61B 5/02

(52) U.S. Cl. CPC A61B 18/18; A61B 2562/028; A61B 5/02; G01N 21/62

(57) ABSTRACT

Described herein are devices and systems configured to detect an energy from a cell, a cell population, and/or a cellular microenvironment and can optionally actuate a cell or population thereof in response to the detection of the energy. Also described herein are methods of operation and use of the devices and systems described herein.

### CROSS-REFERENCE TO RELATED APPLICATIONS

This application claims priority to, and the benefit of, U.S. Provisional Application Ser. No. 62/166,459 filed on May 26, 2015, the entirety of which is incorporated herein by reference.

### STATEMENT REGARDING FEDERALLY SPONSORED RESEARCH OR DEVELOPMENT

This invention was made with government support under grant number 1454555 awarded by the National Science Foundation (NSF). The government has certain rights to the invention.

### BACKGROUND

High throughput in vitro assays can be time consuming and expensive, especially when conducted at a large scale. However, the need for data collected on a large scale has proven necessary when examining drug candidates or other therapies. As such, there exists an urgent and unmet need for improved cell culture systems and devices that can be applied to large scale culture assays and systems.

### SUMMARY

Provided herein are cellular interfacing arrays that can contain a plurality of pixels each of which can contain a sensor that can be configured to detect an energy from a cell, population of cells, or cellular microenvironment and an actuator that can be configured to generate an actuation energy that is capable of actuation the cell or population of cells within responsive proximity to the actuator, where the sensor can be in direct or indirect communication with the actuator. In some embodiments, least one of the pixels of the plurality of pixels of the cellular interfacing arrays can contain at least two actuators. The cellular interfacing arrays can be configured to actuate cells using multiple modalities. In some embodiments, least one of the pixels of the plurality of pixels of the cellular interfacing arrays can contain at least two sensors. The cellular interfacing arrays can be configured to sense multiple modalities In some embodiments, each pixel of the plurality of pixels can contain an operational amplifier. The cellular interfacing arrays having an operational amplifier can be configured using CMOS, where the CMOS can have a feature size of greater than or equal to 130 nm. In some embodiments, each pixel does not contain an operational amplifier. In some embodiments where each pixel does not contain an operational amplifier, the cellular interfacing array can be configured using CMOS, where the CMOS has a feature size of less than 130 nm. The actuation energy can be acoustic, optical, vibrational, thermal, electrical, magnetic, electromagnetic, radioactive, or any permissible combinations thereof. The cellular interfacing arrays can further include one or more substrates. The one or more substrates can be coupled to the cellular interfacing array. The substrate can be a printed circuit board, cell culture container, biocompatible mesh, or combinations thereof. The cellular interfacing arrays can further contain a transmitter and a receiver, wherein the transmitter can be configured to transmit a signal from the cellular interfacing array to a processor or device having a processor, and wherein the receiver can be configured to receive a signal from the processor or the device having a processor. In some embodiments, a transceiver can contain the transmitter and the receiver. In some embodiments, the device having a processor can be a back-end data server. In some embodiments, the plurality of pixels can be heterogeneous.

Also provided herein are cellular interfacing arrays that can contain a plurality of pixels, where each pixel of the plurality of pixels can contain a sensor configured to detect an energy from a cell, population of cells, or cellular microenvironment, and where each pixel of the plurality of pixels does not contain an operational amplifier. Each pixel of the plurality of pixels of the cellular interfacing arrays without an operational amplifier can further contain an actuator configured to generate an actuation energy capable of actuating the cell or population of cells within responsive proximity to the actuator, and wherein the actuator is in communication with the sensor. In embodiments, least one of the pixels of the plurality of pixels of the cellular interfacing arrays without an operational amplifier can contain at least two sensors. The cellular interfacing arrays without an operational amplifier can be configured to sense multiple modalities. The cellular interfacing arrays without an operational amplifier can be configured using CMOS, where the CMOS has a feature size of less than 130 nm. The actuation energy is acoustic, optical, vibrational, thermal, electrical, electromagnetic, radioactive, or any combination thereof.

The cellular interfacing arrays without an operational amplifier can contain one or more substrates, wherein the one or more substrates can be coupled to the cellular interfacing array. The cellular interfacing arrays without an operational amplifier can contain a transmitter and a receiver, wherein the transmitter is configured to transmit a signal from the cellular interfacing array to a device having a processor, and wherein the receiver is configured to receive a signal from the device having the processor. The device having a processor can be a remotely located cloud server. The plurality of pixels of the cellular interfacing arrays without an operational amplifier can be partially heterogeneous.

Also provided herein are medical devices that can contain a cellular interfacing array that can contain a plurality of pixels that can contain a sensor configured to detect an energy from a cell, population of cells, or cellular microenvironment and an actuator configured to generate an actuation energy that is capable of actuation the cell or population of cells within responsive proximity to the actuator, where the sensor can be in communication with the actuator.

Also provided herein are medical devices that can contain a cellular interfacing array that can contain a plurality of pixels, where each pixel of the plurality of pixels comprises a sensor configured to detect an energy from a cell, population of cells, or cellular microenvironment, and where each pixel of the plurality of pixels does not contain an operational amplifier. In embodiments, each pixel of the plurality of pixels can further contain an actuator configured to generate an actuation energy capable of actuating the cell or population of cells within responsive proximity to the actuator, and wherein the actuator is in communication with the sensor.

Also provided herein are methods of detecting an energy from a cell, a population of cells, or a cellular microenvironment that can include the steps of contacting a cell or population thereof with a cellular interfacing array, the cellular interfacing array can contain a plurality of pixels, where each pixel of the plurality of pixels comprises a sensor configured to detect an energy from a cell, population of cells, or cellular microenvironment, and where each pixel of the plurality of pixels does not contain an operational amplifier; and detecting the energy from the cell, population of cells, or cellular microenvironment, wherein the energy from the cell can be detected by the sensor. The methods can further contain the step of actuating the cell or the population of cells by exposing the cell or population of cells to an actuation energy generated by an actuator in a pixel of the plurality of pixels. In some embodiments, at least two energies can be detected from the cell, population of cells, or cellular microenvironment. In some embodiments, the at least two energies can be detected by the same sensor. In some embodiments, the cellular interfacing array can contain at least two sensors and wherein the at least two energies are detected by different sensors. In some embodiments, the different sensors can be contained in the same pixel. In some embodiments, the different sensors are contained in different pixels. In some embodiments, the cellular interfacing array can be configured using CMOS, where the CMOS has a feature size of greater than or equal to 130 nm. In some embodiments, the actuation energy can be acoustic, optical, vibrational, thermal, electrical, magnetic, electromagnetic, radioactive, or any combination thereof. In some embodiments, the methods can further include the step of further transmitting a first signal to a device containing a processing circuitry, wherein the device is configured to receive the first signal. The method can further include the step of receiving a second signal from the device containing processing circuitry, wherein the second signal is received by the cellular interfacing array, wherein the device is configured to transmit the second signal and wherein the receiver is configured to receive the second signal and actuating a cell or population of cells within responsive proximity to the actuator. The method can further include the step of transmitting a first signal to a device containing a processing circuitry, wherein the device is configured to receive the first signal. The methods can further include the step of contacting the cells with a compound of interest.

Also provided herein are methods of detecting an energy from a cell, a population of cells, or a cellular microenvironment and can include the steps of contacting a cell or population thereof with a cellular interfacing array, where the cellular interfacing array can contain a plurality of pixels that can contain comprise a sensor that can be configured to detect an energy from a cell, population of cells, or cellular microenvironment and an actuator that can be configured to generate an actuation energy that is capable of actuation the cell or population of cells within responsive proximity to the actuator, where the sensor is in communication with the actuator, detecting the energy from the cell or population thereof, where the energy is detected by the sensor, and actuating a cell or population of cells that is in responsive proximity to the actuator by generating an actuation energy and exposing the cell or population of cells that are in responsive proximity to the actuator to the actuation energy. In some embodiments, each pixel can contain an operational amplifier. In some embodiments where each pixel contains an operational amplifier, the cellular interfacing array can be configured using CMOS, where the CMOS can have a feature size of greater than or equal to 130 nm. In other embodiments, each pixel of the cellular interfacing array does not contain an operational amplifier. In some of these embodiments, the cellular interfacing array can be configured using CMOS and where the CMOS can have a feature size of less than 130 nm. The actuation energy can be acoustic, optical, vibrational, thermal, electrical, electromagnetic radioactive, or any combinations thereof. The sensor can be in communication with the actuator via a signal processing unit. The signal processing unit can be a server.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 shows a diagram depicting an embodiment of a configuration of the devices and systems described herein and an embodiment of their operation.

FIG. 2 shows embodiments of a cellular interfacing array, where each pixel contains an operational amplifier, at least one sensor, and an actuator.

FIG. 3 shows embodiments of a cellular interfacing array, where each pixel does not contain an operational amplifier but does contain at least one sensor and an optional actuator.

FIG. 4 shows other embodiments of a system containing a cellular interfacing array.

FIG. 5 shows additional embodiments of a system containing a cellular interfacing array as shown in FIG. 2 or 3.

FIG. 6 shows further embodiments of a system containing a cellular interfacing array as shown in FIG. 2 or 3.

FIG. 7 shows embodiments of a cell culture container incorporating a system containing a cellular interfacing array.

FIG. 8 shows embodiments of a medical device incorporating a system containing a cellular interfacing array.

FIGS. 9A-9B show embodiments of the operation of a system containing a cellular interfacing array as shown in FIGS. 2 and 3 and communication between components of the system.

FIG. 10 shows further embodiments of the operation of a system containing a cellular interfacing array as shown in FIGS. 2 and 3 and communication between components of the system.

FIG. 11 shows an embodiment of the use of a medical device incorporating a cellular interfacing array as shown in FIGS. 2 and 3.

FIG. 12 shows embodiments of the operation of a medical device incorporating a system containing a cellular interfacing array as shown in FIG. 11.

FIGS. 13A and 13B shows the temperature profile within a cellular interfacing array after actuation with 1.5 mW RF power at 2.5 GHz.

FIG. 14 shows a circuit diagram and configuration of a cellular interfacing array designed for subcellular resolution, where each pixel does not contain an operational amplifier.

FIGS. 15A-15D show micrographic images of a cellular interfacing array designed for subcellular resolution, where each pixel does not contain an operational amplifier.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of molecular biology, microbiology, nanotechnology, organic chemistry, biochemistry, electrical engineering, synthetic biology, computer science, semiconductors, chemical engineering, and the like, which are within the skill of the art. Such techniques are explained fully in the literature.

### Definitions

As used herein, “about,” “approximately,” and the like, when used in connection with a numerical variable, generally refers to the value of the variable and to all values of the variable that are within the experimental error (e.g., within the 95% confidence interval for the mean) or within +−10% of the indicated value, whichever is greater.

As used herein, “sensor” refers to a transducer that can convert a form of energy into other types of signals, such as an electrical, electromagnetic, or vibrational signals.

As used herein, “actuator” refers to a mechanism or device that supplies and/or transmits an amount of energy and/or signal that stimulates a response and/or the operation of another mechanism or system, biologic or otherwise.

As used herein, “array” refers to an interface having identifiable and discrete pixels and/or pixel groups.

As used herein, “pixel” refers to the basic unit of the cellular interfacing array.

As used herein, “pixel group” can refer to a collection of two or more pixels that are connected electrically, functionally, and/or operationally. A pixel group can include pixels that are adjacent to one another and/or pixels that are not. As such, the arrangement of pixels within a pixel group is not limited to that shown within the figures of the present application.

As used herein, “actuation energy” refers to the energy supplied and/or transmitted by the actuator that stimulates a response and/or the operation of another mechanism or system, biologic or otherwise. As used herein, an actuator can be described in terms of the form of actuation energy it supplies and/or transmits. The actuation energy can be acoustic, optical, vibrational, thermal, electrical, mechanical, radioactive, and any permissible combinations thereof.

As used herein, “communication” refers to the transfer of information and/or energy and/or signals between one or more devices or components thereof. Communication can be physical, electrical, electromagnetic, mechanical, nuclear, atomic, visual, audible, molecular, thermal, fluidic, vibrational, wireless, chemical, and/or magnetic.

As used herein, “responsive proximity” refers to the distance a cell can be from an actuator and still produce a biological response to an actuation energy generated by the actuator.

As used herein, “substrate” refers to a material or layer that underlies or supports an object, or on which some process occurs. The substrate can be solid, porous, mesh, woven, flexible, substantially inflexible.

As used interchangeably herein, “subject,” “individual,” or “patient,” refers to a vertebrate, preferably a mammal, more preferably a human. Mammals include, but are not limited to, murines, simians, humans, farm animals, sport animals, and pets. The term “pet” includes a dog, cat, guinea pig, mouse, rat, rabbit, ferret, and the like. The term “farm animal” includes a horse, sheep, goat, chicken, pig, cow, donkey, llama, alpaca, turkey, and the like.

As used herein, “biocompatible” or “biocompatibility” refers to the ability of a material to be used by in-vitro or in-vivo molecules, cells, tissues or organs that will not affect the natural processes of the in-vitro or in-vivo molecules, cells, tissues or organs or to be used by a patient without eliciting an adverse or otherwise inappropriate host response in the patient to the material or a derivative thereof, such as a metabolite, as compared to the host response in a normal or control patient.

Discussion

Cell culture is used for a variety of applications from cell-based assays for drug discovery to development of cell populations and tissues in regenerative medicine. Cell-based assays are powerful tools to characterize cell or tissue specific physiological behaviors under external biochemical stimuli. External biochemical stimuli can trigger endogenous cellular mechanisms and pathways that can result in a measurable signal or cellular response. Cell-based assays are widely used for large-scale drug screening in the pharmaceutical industry to determine, among other things, potency and toxicity of candidate compounds for drug development.

However, current cell-based assays are conducted on single-modality sensors (e.g. electrical or optical only). As such, current cell-based assays are incapable of capturing the complexity of multi-parameter physiological responses. Sequential transport of cell samples through different sensor platforms results in low throughput and potential abrogation of cell functions. Further, some measurements cannot be performed sequentially. Parallel monitoring of multiple samples with different modalities is subject to cell-to-cell variation and thus increases the materials and expense of such assays.

Current cell culture techniques for regenerative medicine rely heavily on very detailed cell culture methodology developed primarily by trial and error. In short, in most regenerative cell culture protocols a progenitor population of cells is driven down differentiation pathways by adding, for example, growth factors at very specific times, and harvesting cells when they achieve a particular morphology, structure, or express a particular panel of proteins and/or can function a particular way. Protocols have been developed over time by trial and error, where certain cell culture methods are employed and the cell parameters are measured. This is repeated until a population of cells had the morphology, structure, or desired function. Adding to the uncertainty, each cell in a population is capable of spontaneously mutating to a variant, which can taint the entire population. In short, most cell culture protocols result in a highly heterogeneous cell population and cannot prevent undesirable cells within the population or any way to accurately direct the cells down a particular cell lineage. While cells can be sorted by various mechanisms (e.g. fluorescence activated cell sorting (FACS), clonal selection, magnetic-activated cell sorting, and pull down methods), these mechanisms require undesirable cell manipulation and added time and expense. However, no cell culture method or apparatus exists that can provide multi-modal sensing and the capability to actuate a cell or cells within the population on a micron or smaller scale. As a result, in most of clinical centers in US, the stem cell products are often not fully characterized before their implantation for the stem-cell therapy. The inhomogeneity of the cell populations and possible cancerogenic characteristics of the cells have greatly limited the clinical success of the stem-cell therapy and regenerative medicine in practice.

With that said, described herein are devices and systems that can be configured to sense multiple modalities in cells simultaneously and actuate cells autonomously and/or manually through one or more modalities. In some embodiments, the cellular interfacing array can be configured to allow sensing and actuation of cells at the single cell and/or sub-cellular level. Further described herein are methods of sensing multiple sensing modalities and actuating a cell or cells autonomously or manually through one or more actuation modalities. Moreover, methods of using the devices and systems described herein are provided. Other devices, systems, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

Systems and Devices for Cell Sensing and/or Cell Actuation

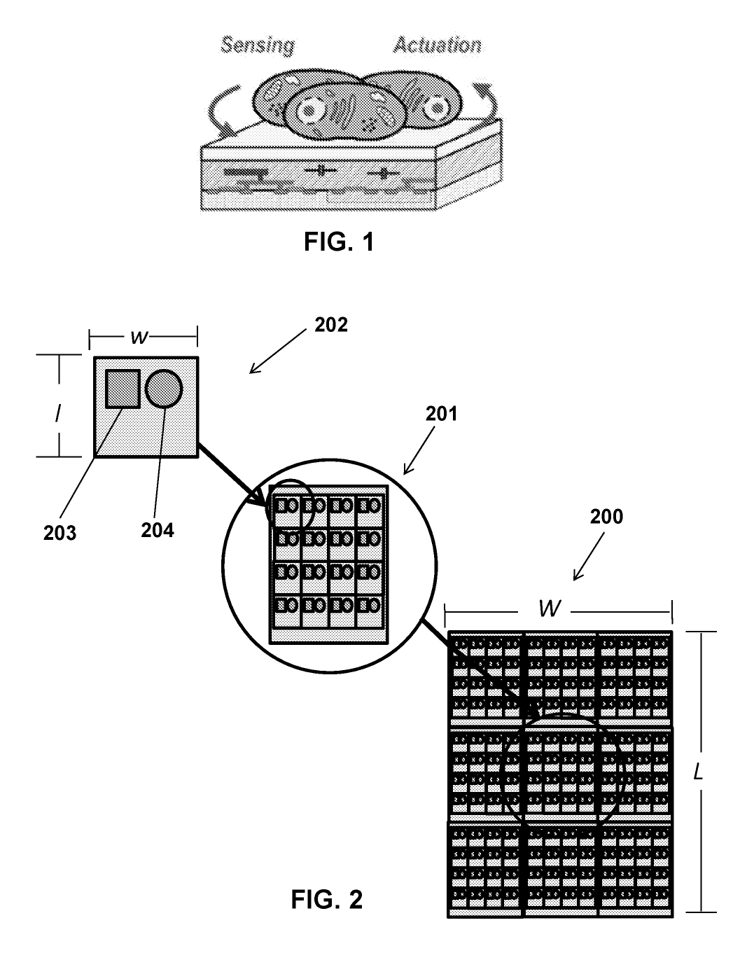

As shown in FIG. 1, the devices and systems described herein can be configured to measure, detect, and/or sense, one or multiple modalities (e.g. energies) of a cell, population of cells, and/or cellular microenvironment, as well as can be optionally configured to actuate a cell or population of cells (either manually or autonomously) in direct contact with or within responsive proximity to a cellular interfacing array having at least one actuator. Co-pending U.S. application Ser. No. 15/050,002 entitled “Multimodality CMOS Cellular interfacing array for Physiological Characterization of Cells” by Wang et al., filed on Feb. 22, 2016, describes multimodality CMOS based cellular interfacing arrays that are configured to sense multiple-modalities from cells. It is worth noting that the array of co-pending U.S. application Ser. No. 15/050,002 focuses on sensing only. Moreover, each pixel of the cellular interfacing arrays of that application contains an operational amplifier (“op-amp”) only implemented using a CMOS process with 130 nm or larger technology nodes or feature size (e.g., 180 nm, 350 nm, etc.). The embodiments of the systems and devices described herein can be configured to contain an op-amp in each pixel or can be configured not to contain an op-amp in each pixel. In those embodiments that are described to contain an op-amp in each pixel, each pixel also contains an actuator, and thus the cellular interfacing array is configured to at least actuate through one or more modalities. Alternatively, in those embodiments that are described to contain an op-amp in each pixel, each pixel can be sensor only but using CMOS process with smaller-than 130 nm technology nodes or feature size. In those embodiments that do not contain an op-amp in each pixel, the cellular interfacing array can be configured to sense one or more modalities and can optionally further contain one or more actuators to actuate through one or more modalities. Embodiments that do not include the op-amp have the advantage of being able to achieve a higher resolution of sensing and/or actuation (depending on the configuration of the pixels) because the electrical components that make up each pixel have a physically smaller footprint. As such, these embodiments can have the additional benefit of providing, in some cases, subcellular resolution. Moreover, embodiments of the systems and devices described herein include multi-modality cellular sensor array designs using CMOS process with CMOS technology nodes (or feature size) below 130 nm (e.g., 90 nm and 45 nm, etc.) and multi-modality sensor/actuator joint array designs with any CMOS technology nodes.

Cellular Interfacing Arrays Configured to Sense Modality(ies) and/or Actuate Cells

Cellular Interfacing Arrays Configured to Sense Modality(ies) and Actuate Cells

With that said, discussion begins with FIG. 2, which shows embodiments of a cellular interfacing array 200 having one or more actuators. The cellular interfacing array 200 can be configured as a microchip. The microchip can have a minimum device feature size (e.g., the minimum CMOS device gate length) ranging from 10 nm to 130 nm or more. In various embodiments, the feature size can range from 14 nm, 20 nm, 30 nm, 40 nm, 45 nm, 50 nm, 60 nm, 65 nm, 70 nm, 80 nm, 90 nm, 100 nm, 110 nm, or 120 nm, to 130 nm or more, including other standard feature sizes. The cellular interfacing array 200 can be configured as or can include microelectromechanical systems (MEMS), chemical field-effect transistors, ion-sensitive filed-effect transistors, enzyme field-effect transistors, metal-oxide semiconductor field effect transistor (MOSFET) complementary metal-oxide-semiconductors (CMOS), compound semiconductor transistors or structures, charge-coupled devices, quad cells, and/or biomedical microelectromechanical systems (Bio-MEMS), graphene structures or transistors, and carbon nanotube structures (CNT). In some embodiments, an actuator can be included in the pixels of a cellular interfacing array. Examples of cellular interfacing arrays can be those described in co-pending U.S. application Ser. No. 15/050,002 entitled “Multimodality CMOS Cellular interfacing array for Physiological Characterization of Cells” by Wang et al., filed on Feb. 22, 2016.

The cellular interfacing array 200 can include one or more pixels 202 (e.g. a plurality of pixels) and/or one or more types of pixels. The cellular interfacing array 200 can have one or more pixel groups (e.g. 201a, 201b, 201c . . . , or collectively 201). One or more pixel groups 201 within the cellular interfacing array 200 can be in direct physical contact with one or more other pixel groups 201. In other embodiments, one or more pixel groups 201 of the cellular interfacing array 200 are not in direct physical contact with one or more other pixel groups 201. In some embodiments, two or more of the pixels or types of pixels can be included in a pixel group 201. The cellular interfacing array 200 can contain at least one pixel 202 that contains at least one actuator 204. The cellular interfacing array 200 can contain at least one pixel 202 that contains at least one sensor 203.

As discussed, the pixels 202 can contain one or more sensors (collectively 203). The sensor(s) 203 can be configured to each individually and/or collectively detect one or more energies from a cell, population thereof, or cellular microenvironment. The number and/or types of sensors in each pixel can range from 0 to 10, or more. The energy can be any form of energy, including but not limited to, thermal energy, electrical energy, magnetic energy, electromagnetic energy, optical energy, vibrational energy, magnetic energy, chemical energy, mechanical energy, and/or elastic energy. The sensor(s) 203 can be configured to directly measure a characteristic of a cell, cell population, cellular microenvironment, and/or biomolecular concentration, or can be configured to indirectly detect a characteristic of a cell, cell population, cellular microenvironment, and/or biomolecular concentration or any combination thereof. The sensor(s) 203 can be configured to convert an energy input into an output signal, such as a voltage, impedance, sound, light, or other signal. The sensor(s) 203 can be biocompatible.

Suitable sensors 203 include, but are not limited to, accelerometers, hygrometer, microphones, chemical sensors, biomolecule sensors, electrochemical gas sensors, electrolyte-insulator-semiconductor sensors, fluorescent chloride sensors, fluorescence resonance energy transfer (FRET)-based sensors, holographic sensors, hydrocarbon dew point sensors, surface acoustic wave sensors, nondispersive infrared sensors, ion selective electrodes, olfactometers, optical sensors, photodiodes, pellistors, potentiometric sensors, zinc oxide nanorod sensors, current sensors, Daly detectors, galvanometers, Hall effect sensors, Hall probes, magnetometers, magnetic field sensors, voltmeters, flow sensors, mass flow sensors, cloud chambers, gyroscopes, altimeters, auxanometers, capacitive displacement sensors, gravimeters, inclinometers, integrated circuit piezoelectric sensors, laser surface velocimeters, linear variable differential transformers, odometers, photoelectric sensors, piezoelectric sensors, position sensors, rate sensors, rotary encoders, rotary variable differential transformers, stretch sensors, ultrasonic thickness gauges, variable reluctance sensors, velocity receivers, colorimeters, contact image sensors, electro-optical sensors, infrared sensors, kinetic inductance detectors, light emitting diode sensors, light addressable potentiometric sensors, fiber optic sensors, optical position sensors, photo detectors, phototransistors, photoionization detectors, photo-electric switches, scintillometers, single-photon avalanche diodes, superconducting nanowire single-photon detectors, transition edge sensors, visible light photon counters, wavefront sensors, barographs, barometers, densitometers, pressure sensors, tactile sensors, bhangmeters, hydrometers, force sensors, level sensors, torque sensors, viscometers, strain gauges, bolometers, microbolometers, bimetallic strips, gardon gauges, heat flux sensors, thermometers, thermistors, pyrometers, proximity sensors, reed switches, and/or photoelastic sensors.

The cellular interfacing array 200 can also contain one or more actuators 204, where the actuator(s) are contained within one or more of the pixels 202. In other words, all, some or only one of the pixels 202 can contain one or more actuators 204. The number of actuators in any given pixel 202 can range from 0 to 10, or more. The actuator(s) 204 each and/or collectively can be configured to generate one or more forms of actuation energies that can actuate a cell or population thereof located within responsive proximity to the actuator through one or more actuation modalities. As shown in FIG. 2, the actuator(s) 204 can be in physically and/or electrically coupled within one or more pixels 202. Suitable actuators can include, without limitation, electrical voltage actuators, electrical current actuators, electromagnetic actuators, mechanical actuators, thermal actuators, ionizing actuators, non-ionizing actuators, vibrational actuators, chemical actuators, atomic actuators, and/or tension actuators. The actuator(s) 204 can be configured to receive one or more signals directly or indirectly from the sensor(s) 204. The actuator(s) 204 can be biocompatible.

In any given cellular interfacing array 200, each pixel 202 can contain the same or different type and/or number of sensors 203 and/or different type and/or number of actuators 204. In further embodiments, some pixels 202 can contain the same type and/or number of sensors 203 and/or actuators 204 while others in the same cellular interfacing array 200 contain a different number and/or type of sensors 203 and/or actuators 204. Stated differently, the cellular interfacing array 200 can be completely homogeneous (every pixel 202 in the cellular interfacing array is identical to each other), completely heterogeneous (no two pixels 202 in the cellular interfacing array are identical to each other), or partially heterogeneous, which can refer to any configuration between (i.e. at least two pixels in the cellular interfacing array are different from each other). For example, some pixels can include a sensor that is configured to measure or detect one sensing modality and other pixels can include sensors with a different modality or even multimodal sensors, while still other pixels may not include any sensors.

The cellular interfacing array 200 can have a length (L), width (W), and a height (or thickness) (H). Although shown as a rectangular array in FIG. 2, it will be appreciated that the cellular interfacing array 200 can be any configuration, including but not limited to a single row of pixels, or any other regular or irregular shape. It will be appreciated that the dimensions of the cellular interfacing array 200 will vary as a function of the pixels 202 and/or pixel groups 201 contained within the cellular interfacing array 200. In some embodiments, the length (L) of the cellular interfacing array 200 can range from about 1 nm to about 1 meter, or more. In some embodiments, the width (W) of the cellular interfacing array 200 can range from about 1 nm to about 1 meter, or more. In some embodiments, the height (H) of the cellular interfacing array 200 can range from about 1 nm to about 500 microns, or more. The cellular interfacing array 200 and components thereof can be biocompatible.

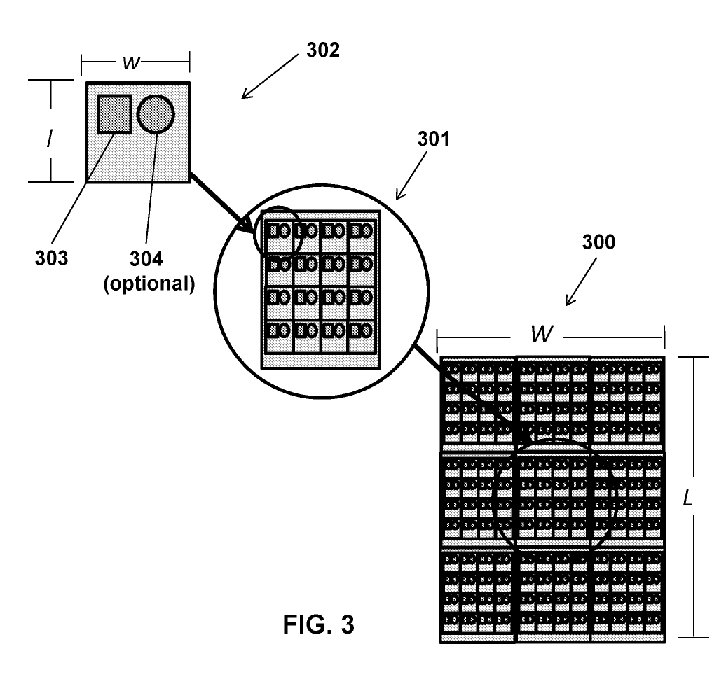

As discussed previously, one or more pixels 202 of a cellular interfacing array 200 configured to sense at least one modality and actuate cells using at least one modality, can include (FIG. 2) or not include an op-amp (FIG. 3) in each pixel. Examples of cellular interfacing arrays having pixels containing op-amps, but are without actuators, are described in co-pending U.S. application Ser. No. 15/050,002 entitled “Multimodality CMOS Cellular interfacing array for Physiological Characterization of Cells” by Wang et al., filed on Feb. 22, 2016. Pixels 202 containing sensor(s) 203, actuator(s) 204, and an op-amp can have a length (L) ranging from about 50 μm to about 100 μm in some embodiments. Pixels 202 containing sensor(s) 203 and actuator(s) 204 that contain an op-amp can have a width (W) ranging from about 50 μm to about 100 μm in some embodiments. The number of pixels 200 containing sensor(s) 203 and actuator(s) 204 that contain an op-amp in a cellular interfacing array 200 can range from about 10 to about 1000 in some embodiments. In some embodiments, the cellular interfacing array 200 having at least one or more actuators 204 and with/without one or multiple in-pixel op-amps can be configured as a CMOS having a CMOS device feature size, i.e., CMOS device gate length, of 130 nm or larger.

As discussed previously, in some embodiments one or more of the pixels 302 of the cellular interfacing array 300 do not contain op-amps but can contain sensor(s) 303, and optionally, actuator(s) 304. Pixels 302 containing sensor(s) 303 and/or actuator(s) 304 that do not contain an op-amp can have a length ranging from about 1 μm to about 50 μm in some embodiments. Pixels 302 containing sensor(s) 303 and/or actuator(s) 304 that do not contain an op-amp can have a length ranging from about 1 μm to about 50 μm in some embodiments. The number of pixels 302 containing sensor(s) 303 and/or actuator(s) 304 that contain an op-amp in a cellular interfacing array can range from 100 to 100,000 in some embodiments. In some embodiments, the cellular interfacing array 200 without sensors 203 and without in-pixel op-amps can be configured as a CMOS having a CMOS device feature size, i.e., CMOS device gate length, of 130 nm or larger.

Cellular Interfacing Arrays Configured to Sense Modality(ies) and Optionally Actuate Cells

As previously discussed, while chip based cell culture sensors have been described, it is challenging to achieve integrated cellular interfacing arrays capable of sensing and/or actuating cells on the subcellular level. One factor that has made it difficult to attain such resolution is the physical size limitations of the components that make up the sensor or actuator arrays. Also provided herein are cellular interfacing arrays that can be configured to sense one or more modalities and can optionally be configured to actuate cells with one or more actuation modalities that are within responsive proximity to the array such that the array can attain single cell and/or sub-cellular resolution. One feature that allows such resolution is not including an op-amp at the pixel-level, which allows the foot print of each pixel and/or feature to be substantially smaller. In further embodiments, transistors in the pixel, e.g., functioning as amplifiers, buffers, or control switches, can be shared among the multiple sensing modalities and/or multiple actuation modalities. This also can facilitate the decreased size of the array.

With that said, discussion continues with regard to FIG. 3, which shows one embodiment of a cellular interfacing array 300 with pixels that do not contain an op-amp. The cellular interfacing array 300 can contain one or more sensors 303, and optionally, one or more actuators 304. The sensor(s) 303 and/or actuator(s) 304 can be contained within pixels. The cellular interfacing array 300 can be configured as a microchip. The microchip can have any feature size, i.e. larger or smaller than 130 nm. In embodiments, the feature size can range from 10 nm, 14 nm, 20 nm, 30 nm, 40 nm, 45 nm, 50 nm, 60 nm, 65 nm, 70 nm, 80 nm, 90 nm, 100 nm, 110 nm, or 120 nm, to 130 nm, or larger feature sizes. The cellular interfacing array 300 can be configured as or can include microelectromechanical systems (MEMS), chemical field-effect transistors, ion-sensitive filed-effect transistors, enzyme field-effect transistors, metal-oxide semiconductor field effect transistor (MOSFET) complementary metal-oxide-semiconductors (CMOS), compound semiconductor transistors or structures, charge-coupled devices, quad cells, and/or biomedical microelectromechanical systems (Bio-MEMS), graphene structures or transistors, and carbon nanotube structures (CNT).

The cellular interfacing array 300 can include one or more pixels 302 (e.g. a plurality of pixels). The pixels 302 can contain one or more sensors (e.g. 303a, 303b, 303c . . . , or collectively 303). The cellular interfacing array 300 can have one or more pixel groups (e.g. 301a, 301b, 301c . . . , or collectively 301). One or more pixel groups 301 within the cellular interfacing array 300 can be in direct physical contact with one or more other pixel groups 301. In other embodiments, one or more pixel groups 301 of the cellular interfacing array 300 are not in direct physical contact with one or more other pixel groups 301. In some embodiments, two or more of the pixels can be included in a pixel group 301. The sensor(s) 303 can be configured to detect an energy from a cell, population thereof, or cellular microenvironment. The energy can be any form of energy, including but not limited to, thermal energy, electrical energy, magnetic energy, electromagnetic energy, optical energy, vibrational energy, magnetic energy, chemical energy, mechanical energy, and/or elastic energy. The cellular interfacing array 300 can contain at least one pixel 302 that contains at least one sensor 303, and optionally, can contain at least one pixel 302 that contains at least one actuator 304.

As discussed, the pixels 302 can contain one or more sensors 303. The sensor(s) 303 can be configured to each individually and/or collectively detect one or more energies from a cell, population thereof, or cellular microenvironment. The number of sensors in each pixel can range from 0 to 10 or more. The energy can be any form of energy including, but not limited to, thermal energy, electrical energy, magnetic energy, electromagnetic energy, optical energy, vibrational energy, magnetic energy, chemical energy, mechanical energy, and/or elastic energy. The sensor(s) 303 can be configured to directly measure a characteristic of a cell, cell population, cellular microenvironment, and/or biomolecular concentration, or can be configured to indirectly detect a characteristic of a cell, cell population, cellular microenvironment, and/or biomolecular concentration or any combination thereof. The sensor(s) 303 can be configured to convert an energy input into an output signal, such as a voltage, impedance, sound, light, or other signal. The sensor(s) 303 can be biocompatible.

Suitable sensors 303 include, but are not limited to, accelerometers, hygrometer, microphones, chemical sensors, biomolecule sensors, electrochemical gas sensors, electrolyte-insulator-semiconductor sensors, fluorescent chloride sensors, fluorescence resonance energy transfer (FRET)-based sensors, holographic sensors, hydrocarbon dew point sensors, surface acoustic wave sensors, nondispersive infrared sensors, ion selective electrodes, olfactometers, optical sensors, photodiodes, pellistors, potentiometric sensors, zinc oxide nanorod sensors, current sensors, Daly detectors, galvanometers, Hall effect sensors, Hall probes, magnetometers, magnetic field sensors, voltmeters, flow sensors, mass flow sensors, cloud chambers, gyroscopes, altimeters, auxanometers, capacitive displacement sensors, gravimeters, inclinometers, integrated circuit piezoelectric sensors, laser surface velocimeters, linear variable differential transformers, odometers, photoelectric sensors, piezoelectric sensors, position sensors, rate sensors, rotary encoders, rotary variable differential transformers, stretch sensors, ultrasonic thickness gauges, variable reluctance sensors, velocity receivers, colorimeters, contact image sensors, electro-optical sensors, infrared sensors, kinetic inductance detectors, light emitting diode sensors, light addressable potentiometric sensors, fiber optic sensors, optical position sensors, photo detectors, phototransistors, photoionization detectors, photo-electric switches, scintillometers, single-photon avalanche diodes, superconducting nanowire single-photon detectors, transition edge sensors, visible light photon counters, wavefront sensors, barographs, barometers, densitometers, pressure sensors, tactile sensors, bhangmeters, hydrometers, force sensors, level sensors, torque sensors, viscometers, strain gauges, bolometers, microbolometers, bimetallic strips, gardon gauges, heat flux sensors, thermometers, thermistors, pyrometers, proximity sensors, reed switches, and/or photoelastic sensors.

The cellular interfacing arrays 300 can also optionally contain one or more actuators 304 where the actuator(s) are contained within one or more of the pixels 302. In other words, all, some or only one of the pixels 302 can contain one or more actuators 304. The number of actuators in any given pixel 302 can range from 0 to 10, or more. The actuator(s) 304 each and/or collectively can be configured to generate one or more form(s) of actuation energy that can actuate a cell or population thereof located within responsive proximity to the actuator through one or more actuation modalities. As shown in FIG. 3, the actuator(s) 304 can be in physically and/or electrically coupled within one or more pixels 302. Suitable actuators can include, without limitation, electrical voltage actuators, electrical current actuators, electromagnetic actuators, mechanical actuators, thermal actuators, ionizing actuators, non-ionizing actuators, vibrational actuators, chemical actuators, atomic actuators, and/or tension actuators. The actuator(s) 304 can be configured to receive one or more signals directly or indirectly from the sensor(s) 304. The actuator(s) 304 can be biocompatible.

In any given cellular interfacing array 300, each pixel 302 can contain the same or different type and/or number of sensors 303 and/or actuators 304. In further embodiments, some pixels 302 can contain the same type and/or number of sensors 303 and/or the same type and/or number of actuators 304, while others in the same cellular interfacing array 300 can contain a different number and/or type of sensors 303 and/or a different number and/or type of actuators 304. Stated differently, the cellular interfacing array 300 can be completely homogeneous (every pixel 302 in the cellular interfacing array is identical to each other), completely heterogeneous (no two pixels 302 in the cellular interfacing array are identical to each other), or partially heterogeneous, which can refer to any configuration including combinations of the same and different pixels (i.e. at least two pixels in the cellular interfacing array are different from each other). For example, some pixels can include an actuator that is configured to provide a single activation and other pixels can include multimodal actuators or multiple actuators, while still other pixels may not include any actuators. In some embodiments, some pixels can include both actuator(s) and sensor(s), while other pixels can include either one or more actuator(s) or one or more sensor(s), but not both, and still other pixels may not include either an actuator or sensor.

Pixels 302 without an op-amp can have a length (L) ranging from about 1 μm to about 50 μm in some embodiments. Pixels 302 without an op-amp can have a width (W) ranging from about 1 μm to about 50 μm in some embodiments. The number of pixels 302 containing sensor(s) 303 but are without an op-amp in a cellular interfacing array can range from about 100 to about 100,000 in some embodiments.

The cellular interfacing array 300 can have a length (L), width (W), and a height (thickness) (H). Although shown as a rectangular array in FIG. 3, it will be appreciated that the cellular interfacing array 300 can be any configuration, including but not limited to a single row of pixels, or any other regular or irregular shape. It will be appreciated that the dimensions of the cellular interfacing array 300 will vary as a function of the pixels 302 and/or pixel groups 301 contained within the cellular interfacing array 300. In some embodiments, the length (L) of the cellular interfacing array 300 can range from about 1 nm to about 1 meter or more. In some embodiments, the width (W) of the cellular interfacing array 300 can range from about 1 nm to about 1 meter or more. In some embodiments, the height (H) of the cellular interfacing array 300 can range from about 1 nm to about 500 microns or more. The cellular interfacing array 300 and components thereof can be biocompatible.

Systems Containing Cellular Interfacing Arrays Configured to Sense Cell Modality(ies) and/or Actuate Cells

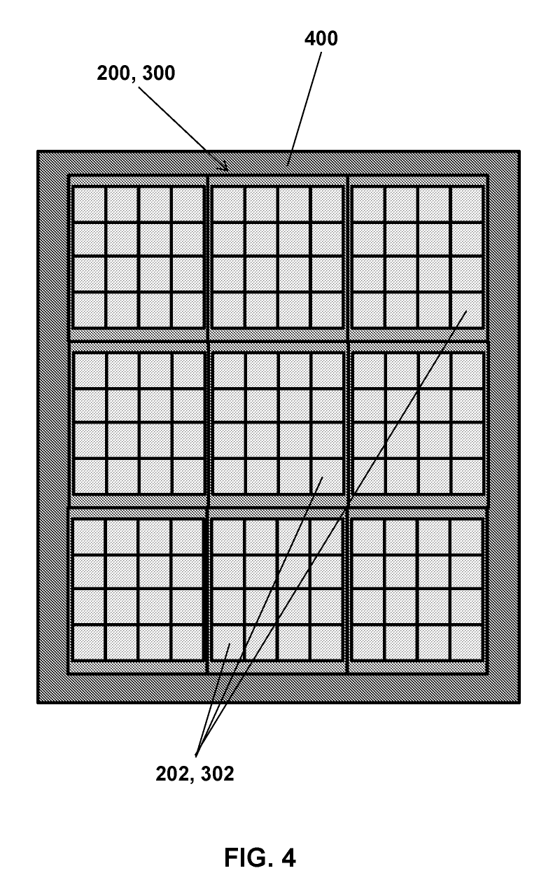

Also described herein are systems that contain one or more cellular interfacing arrays 200, 300 as described elsewhere herein. As shown in FIG. 4, the system can include a substrate 400 that is coupled to the cellular interfacing array(s) 200, 300. The substrate can be solid, semi-solid porous, mesh, woven, laminate, flexible, inflexible, absorbent, transparent, opaque, semi-transparent malleable, hygroscopic, non-hygroscopic, conductive, an insulator, elastic, non-elastic, inorganic, organic, natural, synthetic or any permissible combination thereof. One or more circuits (e.g., processing circuits) can be integrated within and/or coupled to the substrate 400. The circuit(s) can be, without limitation, electrical circuits, optical circuits, electromagnetic circuits, wireless circuits, and chemical circuits. The circuits can be coupled to the cellular interfacing array. The circuit(s) can be in communication with the cellular interfacing array 200, 300 components thereof (including actuator(s) 204, 304 and sensor(s) 202, 302). Embodiments with substrates 400 are discussed in greater detail below. As described in greater detail below, the substrate 400 can be a printed circuit board. In some embodiments, the substrate 400 can be a biocompatible mesh.

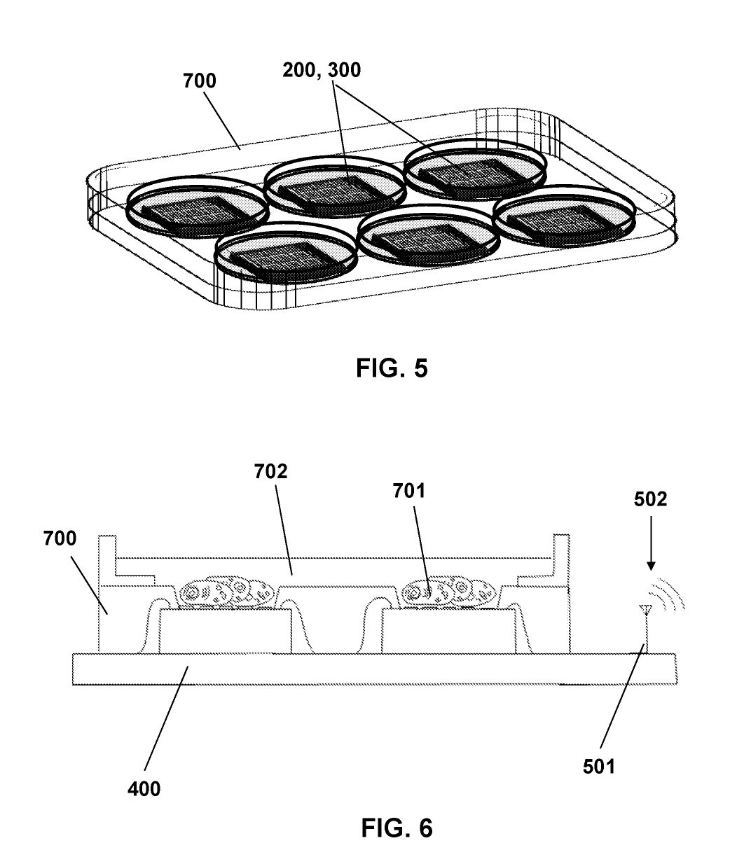

The substrate 400 and components thereof can be biocompatible. The substrate 400 or any component thereof (including any circuits) can be made out of any suitable material or combination of suitable materials. Suitable materials include, but are not limited to metals, metal alloys, nanomaterials (including but not limited to fullerenes, carbon nanotubes, and nanocrystals), ceramics, glass, polymers (including but not limited to plastics), biomaterials (such as, without limitation, bone, chitosan, alginate, cartilage, and hydroxyapatite), conductive materials, insulator materials, semiconductive materials, magnetic materials, inorganic, organic, natural, synthetic, any composites thereof, and/or any permissible combination thereof. In some embodiments, the substrate 400 is a printed circuit board. In some embodiments (e.g. FIG. 5), the substrate can be a cell culture or tissue culture container (e.g. dish, flask, plate, multi-well plate). In some embodiments, the cellular interfacing array 200, 300 can be coupled to more than one substrate 400. For example, the cellular interfacing array 200, 300 can be coupled to a printed circuit board and a cell culture plate (See e.g. FIG. 5).

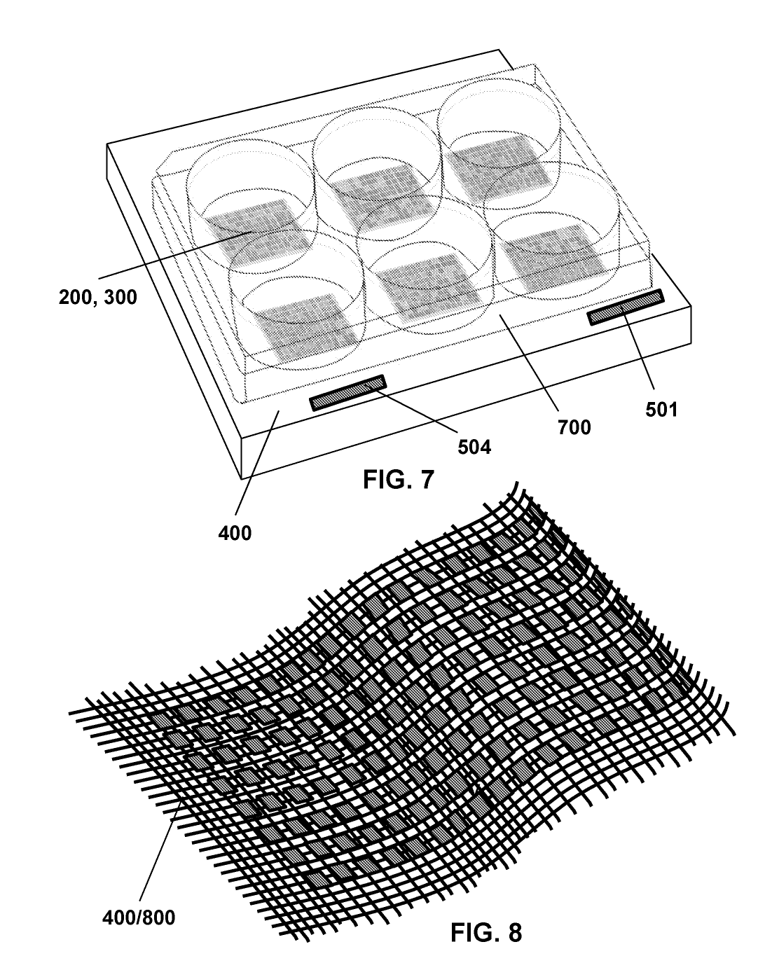

FIGS. 5-7 show embodiments of the cellular interfacing array 200, 300 and systems containing the cellular interfacing arrays 200, 300 described herein that can be incorporated into cell culture containers and/or medical devices. As shown in FIGS. 5-8, the systems described above can be incorporated into a cell culture container 700 (e.g., a multi-well or single-well cell culture plate) having any number of wells. Although FIGS. 5 and 7 show a 6-well cell culture plate, it will be appreciated that the cell culture container 700 can be any cell or tissue culture plate, multi-well plate, vessel, flask, or any other culture container having the same or different number of wells. As illustrated in FIG. 6, cells 701 can be cultured within a media 702 and come in contact with or be in proximity to the cellular interfacing array 200, 300. The cell culture container 700 and cellular interfacing array 200 can be coupled to an additional substrate 400, which can be (for example) a printed circuit board. The substrate 400 can be in communication with or coupled to processing circuitry containing a processor and memory or a device containing a processor and memory. It will be understood that where reference is made in this application to processing circuitry, it is implied that the processing circuitry contains a processor and memory. In some embodiments, the substrate 400 can contain one or more transmitter/transceiver 501 and/or one or more receiver/transceiver 504 (e.g., FIG. 7), where the transmitter 501 and/or transceiver 504 can be configured to transmit a signal (e.g., 502, FIG. 8) to processing circuitry or a device comprising processing circuitry and the receiver/transceiver 504 can be configured to receive a signal (e.g., 503, FIG. 8) from processing circuitry or a device comprising processing circuitry. The transmitter 501 and receiver 504 (or transceiver) can communicate with cloud servers or any back-end data servers. The transmitter/transceiver 501 can be configured transmit a signal (e.g. an energy) to a receiver/transceiver 504 that can be coupled to the cellular interfacing array 200, 300, which can sense and/or stimulate the biological samples through one or more sensing/stimulation modalities. The receiver/transceiver 504 can be configured to obtain a signal from a cellular interfacing array 200, 300, or a component thereof (such as, e.g., a sensor 203, 303). After obtaining a signal from the cellular interfacing array 200, 300, the transmitter/transceiver 501 and/or receiver/transceiver 504, can transmit a signal to processing circuitry or a device comprising processing circuitry. Similarly, the transmitter/transceiver 501 and/or receiver/transceiver 504, can receive a signal from processing circuitry or a device comprising processing circuitry, and the received signal will be relayed to the a cellular interfacing array 200, 300, or a component thereof (such as, e.g., a sensor 203, 303) to (for example) control their operation, set their configurations, and determine the actuation signals/modalities to be applied to the sample. The antennas for the wireless signal transmitting/receiving can be implemented either on the printed circuit board, cell culture container, substrate, device, cellular interfacing array, any composites thereof, and/or any permissible combination thereof.

As shown in FIG. 8, the substrate 400 can be a fabric and/or mesh 800. In some embodiments, the fabric and/or mesh 800 can be a biocompatible fabric or mesh and/or can contain electrically conductive circuits. In some embodiments the fabric and/or mesh 800 can be electrically conductive. The fabric and/or mesh 800 containing a one or more cellular interfacing arrays 200, 300 as described herein can be implantable.

The cellular interfacing array 200, 300 and systems thereof described herein can be incorporated into a medical device. The medical device can be biocompatible. The medical device can be implantable. The medical device can be, without limitation, a catheter, a stent, a surgical mesh, a fixation device, a fusion device, a subperiosteal implant, a prosthesis, a tube, a shunt, a plate, or a spinal implant. Other suitable medical devices can be used as will be appreciated by those of skill in the art.

The cellular interfacing array 200, 300 and systems thereof described herein can be incorporated as a field-deployable device for interfacing with the nearby cellular environment. Such devices can be biocompatible. Such devices can be deployed in cell culture reactors for large-volume cell manufacturing. The devices can be powered using wireless power transfer, energy harvesting, or battery.

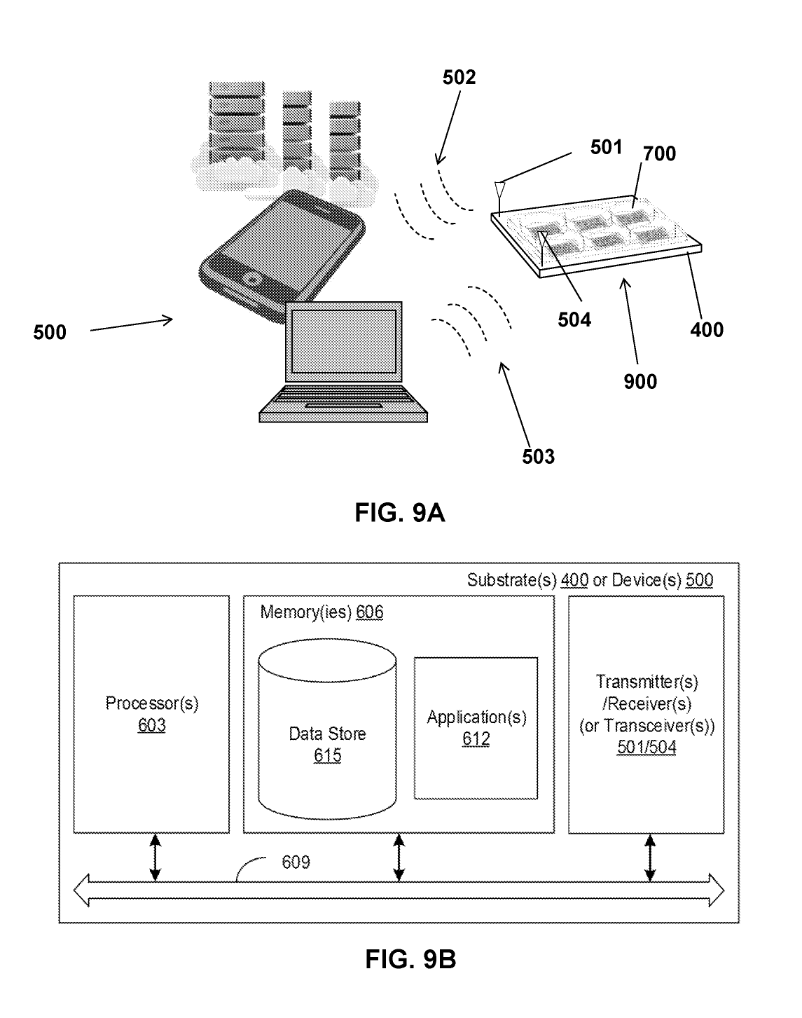

The system containing a cellular interfacing array 200, 300 and/or any component thereof can be coupled to and/or be in communication with a device comprising a processor and/or processing circuitry 500. Such devices include without limitation, computers, smartphones, severs, tablets, digital assistants, smart and smart watches, cloud servers, and any back-end servers. As shown in FIG. 9A, the system 900 containing the cellular interfacing array(s) 200, 300 can be wirelessly and/or non-wirelessly coupled to and/or in communication with the device 500 comprising a processer and/or processing circuitry. The system 900 containing the cellular interfacing array 200, 300, can contain one or more transmitters/transceivers 501 or transceivers 504 that can be configured to transmit signals 502. The signals 502 can be received by a receiver/transceiver 504 and can be processed by processing circuitry such as processing circuitry on the device 500 and/or the substrate 400 and/or cellular interfacing array 200, 300. The device 500 comprising processing circuitry can be configured to transmit signals 500 to the system 900 containing the cellular interfacing array 200, 300 and/or any component thereof, where the system containing a cellular interfacing array 200, 300 and/or any component thereof, where one or more receivers/transceivers 504 are configured to receive the signal 503. The system containing a cellular interfacing array 200, 300 and/or any component thereof can contain one or more receivers 504 configured to receive an energy 503 from the processor and/or device containing a processor 500. Note that although wireless signal transmission is shown as an example, the embodiment of signal transmission can be based on various means, including electromagnetic wave propagation, electromagnetic conduction through wires or waveguides, or optical links.

With reference now to FIG. 9B, shown is a schematic block diagram illustrating an example of a substrate 400, device 500, or the proposed cellular interfacing array. The substrate 400 and/or device 500 can include at least one processor circuit, for example, having a processor 603 and a memory 606, both of which are coupled to a local interface 609. To this end, the device 500 may comprise, for example, at least one server computer, mobile computing device (e.g., smart phone, tablet, laptop computer) or like device. The local interface 609 may comprise, for example, a data bus with an accompanying address/control bus or other bus structure as can be appreciated. The substrate 400 and/or device 500 can also include one or more transmitter 501, one or more receiver 504, and/or one or more transceiver to facilitate wired or wireless communications between the substrate 400 and device 500. For example, if the cellular interfacing array is implemented as CMOS chips, all of these functionalities of the system can be integrated on the same CMOS chip. This can result in minimizing the cost of the materials (e.g. board, substrate) and packaging.

Stored in the memory 606 are both data and several components that are executable by the processor 603. In particular, stored in the memory 606 and executable by the processor 603 are applications 612 for implementing the communication of sensor data and operation of actuators as discussed, and potentially other applications. Also stored in the memory 606 may be a data store 615 including, e.g., collected sensor data. In addition, an operating system may be stored in the memory 606 and executable by the processor 603. It is understood that there may be other applications that are stored in the memory and are executable by the processor 603 as can be appreciated.

Where any component discussed herein is implemented in the form of software, any one of a number of programming languages may be employed such as, for example, C, C++, C#, Objective C, Java®, JavaScript®, Perl, PHP, Visual Basic®, Python®, Ruby, Delphi®, Flash®, or other programming languages. A number of software components are stored in the memory and are executable by the processor 603. In this respect, the term “executable” means a program file that is in a form that can ultimately be run by the processor 603. Examples of executable applications or programs may be, for example, a compiled program that can be translated into machine code in a format that can be loaded into a random access portion of the memory 606 and run by the processor 603, source code that may be expressed in proper format such as object code that is capable of being loaded into a random access portion of the memory 606 and executed by the processor 603, or source code that may be interpreted by another executable program to generate instructions in a random access portion of the memory 606 to be executed by the processor 603, etc. An executable program may be stored in any portion or component of the memory including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory is defined herein as including both volatile and nonvolatile memory and data storage components. Volatile components are those that do not retain data values upon loss of power. Nonvolatile components are those that retain data upon a loss of power. Thus, the memory 606 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 603 may represent multiple processors 603 and the memory 606 may represent multiple memories 606 that operate in parallel processing circuits, respectively. In such a case, the local interface 609 may be an appropriate network that facilitates communication between any two of the multiple processors 603, between any processor 603 and any of the memories 606, or between any two of the memories 606, etc. The processor 603 may be of electrical or of some other available construction.

Although portions of the applications 615, and other various systems described herein may be embodied in software or code executed by general purpose hardware, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits having appropriate logic gates, or other components, etc. Such technologies are generally well known by those skilled in the art and, consequently, are not described in detail herein.

The applications 615 can comprise program instructions to implement logical function(s) and/or operations of the system. The program instructions may be embodied in the form of source code that comprises human-readable statements written in a programming language or machine code that comprises numerical instructions recognizable by a suitable execution system such as a processor in a computer system or other system. The machine code may be converted from the source code, etc. If embodied in hardware, each block may represent a circuit or a number of interconnected circuits to implement the specified logical function(s).

Also, any logic or application 615 described herein that comprises software or code can be embodied in any non-transitory computer-readable medium for use by or in connection with an instruction execution system such as, for example, a processor 603 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Some or all of the transmitter, receiver, antenna (if for wireless connectivity), processors, controls, analog-to-digital convertors, digital-to-analog convertors, and memory blocks or any other components can be on the cellular interfacing array chip, substrate, printed circuit board, cell culture container, mesh, external devices, or combination thereof.

Methods of Multimodal Cell Sensing and Cell Actuation and Feedback Operation

The systems and devices described herein can sense one or multiple modalities from cells and/or actuate the cell(s) using one or more modalities. Sensing and/or actuation and other operations can be executed concurrently, sequentially, or in any combination thereof. The systems and devices described herein can be configured to detect one or more energies from a cell, population of cells, and/or cellular microenvironment and can optionally actuate a cell or population of cells that are in responsive proximity to the cellular interfacing array through one or more energies. This can be done autonomously (without human intervention between the step of sensing and the step of actuating) or manually (with human intervention between the step of sensing and the step of actuating). Where more than one energy from a cell, population of cells, and/or cellular microenvironment is detected, this can also be referred to as multimodal sensing.

Generally, after an energy is detected from a cell, population of cells, and/or cellular microenvironment, the sensor can transmit a signal to an actuator that is in communication with and/or coupled to the sensor. The signal can be transmitted by the sensor directly to an actuator. The signal can be transited by the sensor indirectly to an actuator. It will be appreciated that indirect transmission of a signal from a sensor to an actuator can refer to the signal from the sensor being transmitted to processing circuitry and then being processed by processing circuitry (comprising, e.g., processor(s) and/or memory), before being sent to the actuators. The processing circuitry can be on the cellular interfacing array chip, substrate, board, cell culture plates, and/or external devices with wired or wireless connections. In some implementations, the signal from the sensor(s) may be processed by circuitry distributed between different locations or may be initially processed by circuitry at a first location and then transmitted for further processing by circuitry at a second location. For example, sensor signals may be preprocessed by circuitry on the cellular interfacing array chip and communicated to processing circuitry on the substrate and/or external device for further processing before sending a control signal to the actuator.

The actuator can generate one or more forms of actuation energy through one or more actuation modalities. When more than one actuation energy is implemented, this can also be referred to as multimodality actuation. This can be in response to receiving a control signal, either directly or indirectly, from the sensor. The actuation energy(ies) can stimulate one or more biological response(s) within the cell or population or cellular microenvironment thereof that are within responsive proximity to the actuator. The actuation energy(ies) can also alter the cell environment through one or more actuation modalities, e.g., electrical, mechanical, chemical, thermal, to indirectly stimulate the cell or population thereof that are within responsive proximity to the actuator. The biological response can include, but is not limited to, death, a modification in a cellular process (such as, e.g., a change in gene and/or protein expression or metabolic process), cellular action potential, and cellular migration behaviors. This can result in maintaining a population of cells that have a homogenous or a desired phenotype and/or functional characteristics. This can also allow for control of differentiation of cells. This can also be used to characterize cells or cellular responses under controlled and/or synchronized stimuli or in controlled and/or synchronized environment.

In some embodiments, the actuation modality can be a radio-frequency (RF) electromagnetic field applied by the actuator(s) or actuator array. The RF electromagnetic field can cause heat generation and temperature increase in the medium or samples due to the increased dielectric loss of water contents at RF frequencies. The actuator(s) or actuator array can be designed and configured so that only one or multiple selective parts of the medium or samples receives such RF-mediated heat generation at specific time. This spatiotemporally controlled RF-mediated heating actuation can be used to apply stress to the cells/tissues, ablate and/or kill the cells and/or tissues, and/or alter the chemical and/or biochemical reactions' characteristics and/or local chemical and/or biochemical environment for the specific region at specific time. In some embodiments, the actuator(s) and actuator array for this RF-mediated heating actuation can be implemented using CMOS platforms. The related on-CMOS circuits include but are not limited to RF signal generator, filter, multiplexer, amplifier, buffer, and RF power amplifier. In some embodiments, the RF power amplifier can be Class-D RF power amplifier to achieve broadband rail-to-rail output within an ultra-compact form-factor. In some embodiments, the signal from the sensor can be transmitted to processing circuitry comprising a processor and memory or a device comprising processing circuitry comprising a processor and memory. The processor can execute a logic function, application and/or program which can be stored in memory and determine if actuation in any pixel is needed. If the processor determines that actuation in any pixel is needed, the processor can transmit or direct the transmission of a signal to an actuator, which in turn can generate an actuation energy. In some embodiments, the device 500 comprising processing circuitry can also have and/or be coupled to a data storage device or system. As such, in some embodiments, information from the sensor can be obtained and stored for analysis. In some embodiments, the device 500 comprising the processing circuitry analyzes the data and determines if actuation in any pixel is desired by, for example, comparing data to predetermined (or preprogrammed) amounts, values, and/or thresholds and determining if the data value is in a range that commands actuation.

In some embodiments, an operator can analyze the data by comparing the data to for example, comparing data to predetermined (or preprogrammed) amounts, values, and/or thresholds and determining if actuation in any pixel is desired. If the operator determines that actuation is desired, the operator can interact with the processor and/or cellular interfacing array to direct transmission of a signal to an actuator, which can in turn generate an actuation energy. The operator can also manually initiate analysis using a logic function, application, or program that is executed by a processor and stored in memory on the device 500.

Methods of Using the Devices and Systems for Multimodal Cell Sensing and Cell Actuation

The systems and devices described herein which can be configured to detect one or more energies from a cell, population of cells, and/or cellular microenvironment and optionally actuate a cell or population of cells that are in responsive proximity to one or more actuator(s) through one or more actuation modalities can be used in a wide variety of applications from compound screening assays, to biomedical and regenerative engineering, to medical treatments, and to environment monitoring.

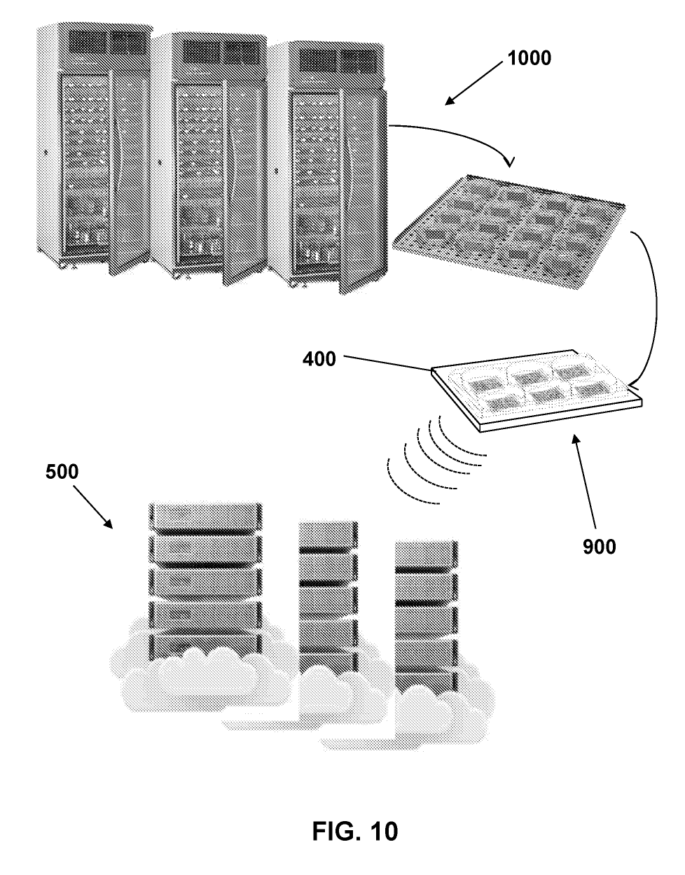

As shown in FIG. 10, the systems and devices described herein can be utilized in small and large scale in-vitro assay systems 1000. The systems and devices described herein can have the advantage of being able to autonomously sense one or multiple cellular responses (e.g. can be single modal or multimodal) and can optionally actuate the cells when required on any scale through one or multiple actuation signals (e.g. can be single modal or multimodal). In use, cell culture containers, as described in detail with respect to FIGS. 5-8, containing cells (e.g. heart cells, neuronal cells, or any other cells) can be cultured, for example, under experimental culture conditions or with test compounds (e.g. such as during a typical drug screening assay). As the cells respond to the experimental culture conditions or candidate compounds, the cellular interfacing array(s) 200,300 in the system can detect one or more energies from a cell, population of cells, and/or cellular microenvironment and directly or indirectly send a wireless signal to a processing circuitry or a device containing processing circuitry. The system can optionally store and/or analyze the sensor data and optionally autonomously actuate one or more cells within the cell culture container by selectively activating one or more actuators. In some embodiments, an operator can intervene between the step of sensing and actuating and manually determine if actuation is needed. In this way, the cells can remain undisturbed during the assay, yet data on multiple modalities can be recovered. Further, the cell population can be autonomously or manually controlled by closed loop multi-modality cellular sensing, signal processing/computation/memory-storage, and multi-modality cellular actuation.

In stem cell culture and manufacturing, the systems described herein can be used to control a cell population and stimulate differentiation down particular cell lineages and pathways. In some embodiments, pluripotent, multipotent, or totipotent cells can be cultured on a cell culture container containing a system, device described herein, or a culture reactor. The state of the differentiated cells can be real-time monitored and holistically characterized using the multi-modality sensing and signal processing/computation/memory-storage. The sensor data can be monitored manually by an operator or autonomously by a processing circuitry. The cells can be actuated to guide or facilitate the cells down particular differentiation pathways, as well as to ablate and eliminate cells that differentiate down an undesired pathway. In this same manner, differentiated cells may be reprogrammed back to a pluripotent state by selectively actuating the cells until induced pluripotent stem cells are generated.

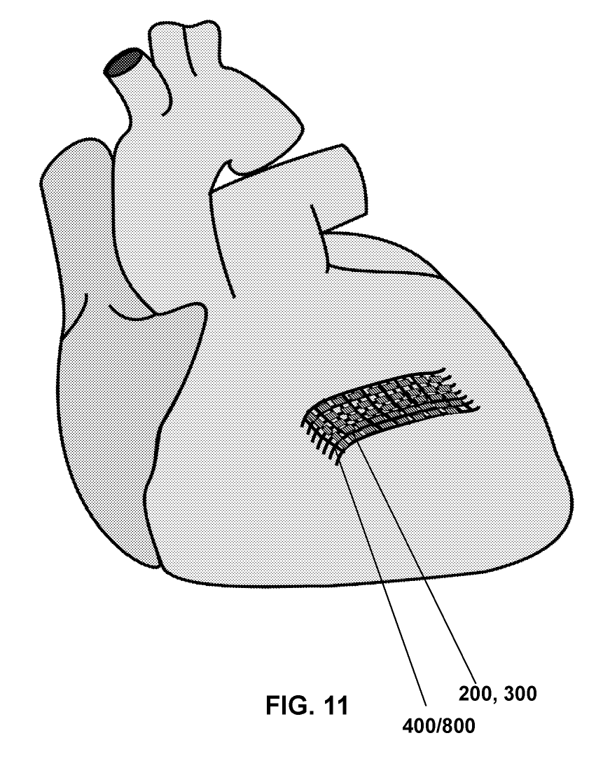

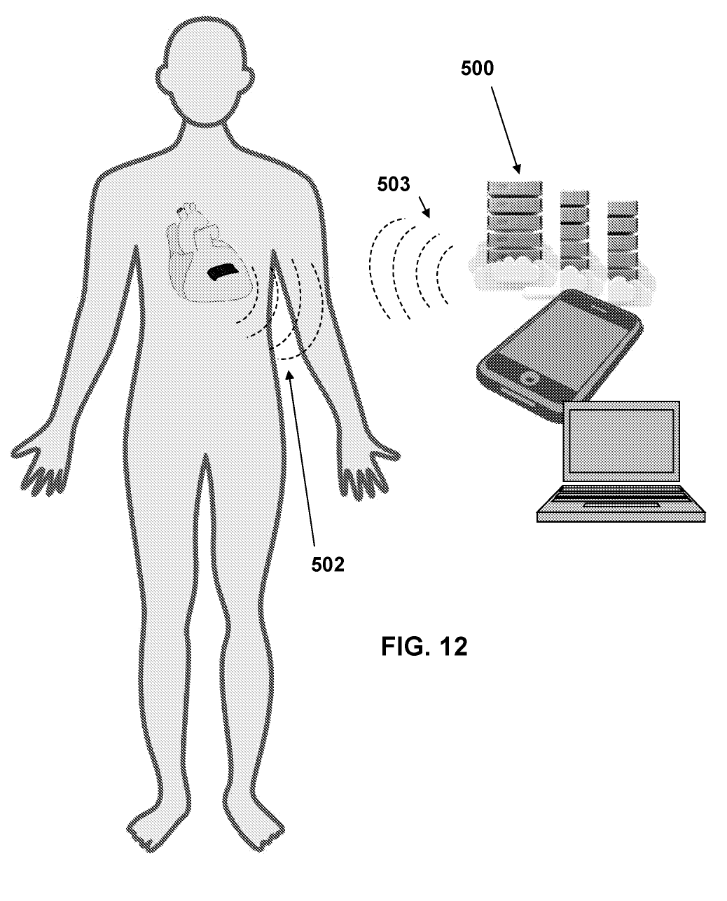

As shown in FIGS. 11-12, the systems and devices described herein can be implanted into a subject in need thereof. In some diseases, injuries, or disorders, it is desirable to selectively ablate or control cells within the body. The cells that can be sensed, characterized, and/or ablated, can be for example, heart cells, neuronal cells, brain cells, spinal column cells, muscle cells, fat cells, liver cells, kidney cells, pancreatic cells, and/or tumor cells. As a non-limiting example, a mesh or other medical device that incorporates a cellular interfacing array as described herein can be implanted to a heart with an electrical conductivity disorder (e.g. an arrhythmia). By autonomously or manually actuating cells that have the abnormal conductivity, the abnormal cells can be made to function like normal cells. The system can be configured to transmit sensor data to a processor or device outside the body. In other embodiments, the processor or device containing the processor can be contained within the body.

In another non-limiting example, the mesh or other medical device containing a cellular interfacing array 200, 300 as described herein can be implanted over an organ or tissue containing tumor cells. Using this system, abnormal, such as tumor cells can be selectively detected, monitored, and/or ablated at a high spatial and/or time resolution.

In another non-limiting example, the mesh or medical device containing a cellular interfacing array 200, 300 as described herein described herein can be implanted into an organ or other tissue in the case for stem cell therapy. The injected cell products differentiated from stem cells in the organ or tissue can be first monitored by the system, and then the abnormal cells can be identified, characterized, and/or ablated to avoid unwanted cell growth, such as cancer. In this way, stem cell therapy can be performed without cancerogenic side-effects.

In another non-limiting example, the mesh or medical device containing a pixelated electric interface described herein can be implanted into an organ or other tissue. Stem cells or other partially differentiated cells can be stimulated down a desired differentiation pathway. In this way enhanced tissue repair and/or regeneration could occur.

In another non-limiting example, the mesh or medical device containing a pixelated electric interface described herein can be deployed in cell culture reactors for large-volume cell manufacturing. One or more devices can be deployed in the cell culture reactors. In this way, the states of the differentiated cells during the large-volume cell manufacturing can be real-time monitored and holistically characterized using the multi-modality sensing and signal processing/computation/memory-storage.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure.

### Example 1

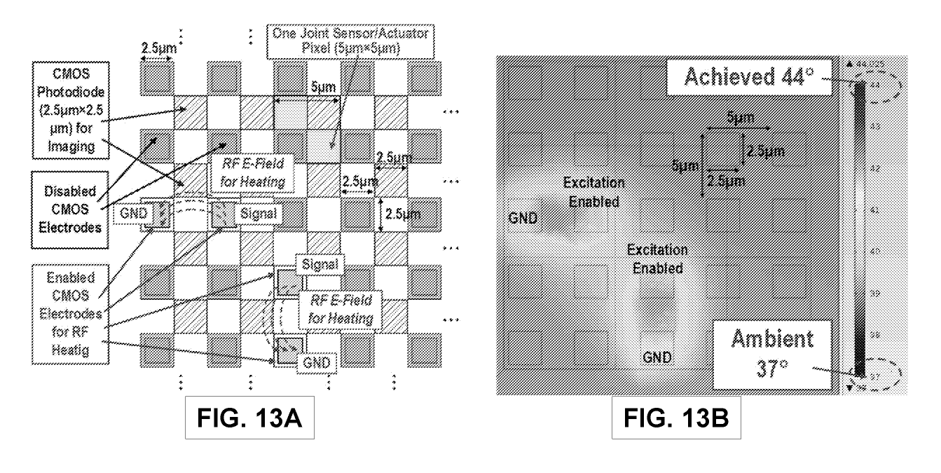

As shown in FIGS. 13A-13B, the cellular interfacing array containing one or more cellular sensors and one or more actuators. The actuators can ablate cells within responsive proximity using radio-frequency (RF) thermal modulation or thermal ablation.

The actuator can contain a compact RF power amplifier, e.g., class-D power amplifier, which amplifies the RF actuation signal to the appropriate power level. The amplified RF actuation signal can be coupled to the nearby cell culture medium and cells, which thus generates a distribution of RF electrical, magnetic, and/or electromagnetic field. Since water experiences substantial dielectric loss at RF frequencies (hundreds of MHz to GHz), the RF actuation signal can be largely dissipated based on its field distribution. Such dissipated RF energy thus leads to the heat generation in the nearby cell culture medium and cells. The generated heat performs thermal modulation of the nearby cell culture medium and cells. Such thermal modulation can alter the action potential generation and conduction in neurons or neuron networks, e.g., suppressing the neuron activities or suppressing the neuron junctions. Such thermal modulation also can alter the local biological, chemical, or biochemical environment by changing the local biological, chemical, or biochemical reactions, e.g., equilibrium points and/or reaction constants. At a certain RF actuation power level, the generated heat can apply controlled stress on the nearby cells or even ablate the nearby cells. With the array configuration, multiple actuators can be enabled with different amplitudes and phases relations, and thus an arbitrary spatial (e.g., a few microns) and temporal (e.g., a few nano-seconds) distribution of the RF thermal modulation or thermal ablation can be generated in a programmable and reconfigurable fashion. The cell culture medium and cells can be thermally actuated with a high spatiotemporal resolution. Low-frequency (below hundreds of MHz) or DC electrical field also can be generated to modulate nearby cell culture medium and cells, including electroporation, manipulation, excitation, and even killing the cells.

### Example 2

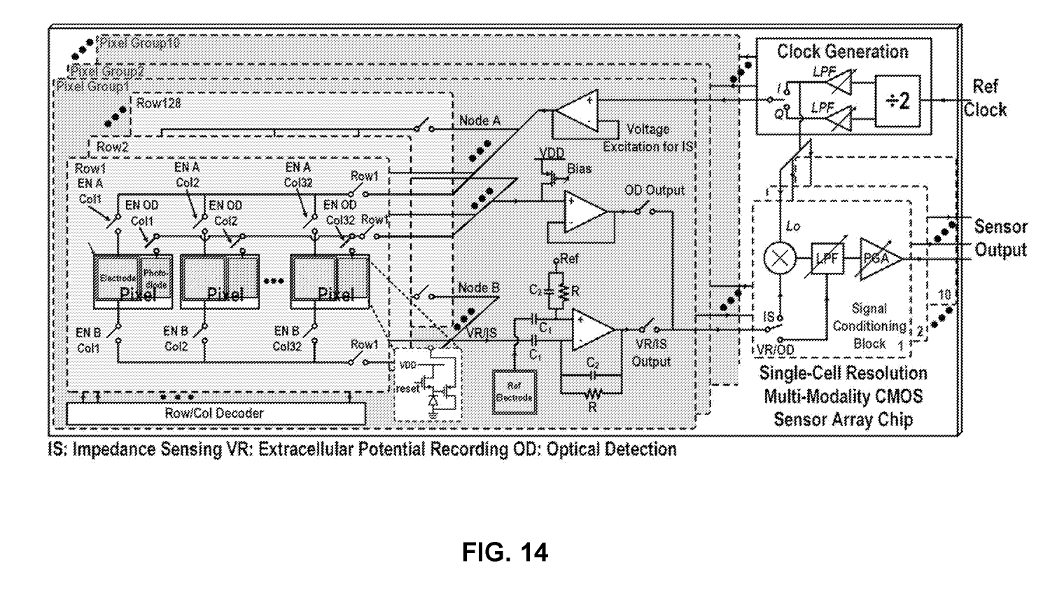

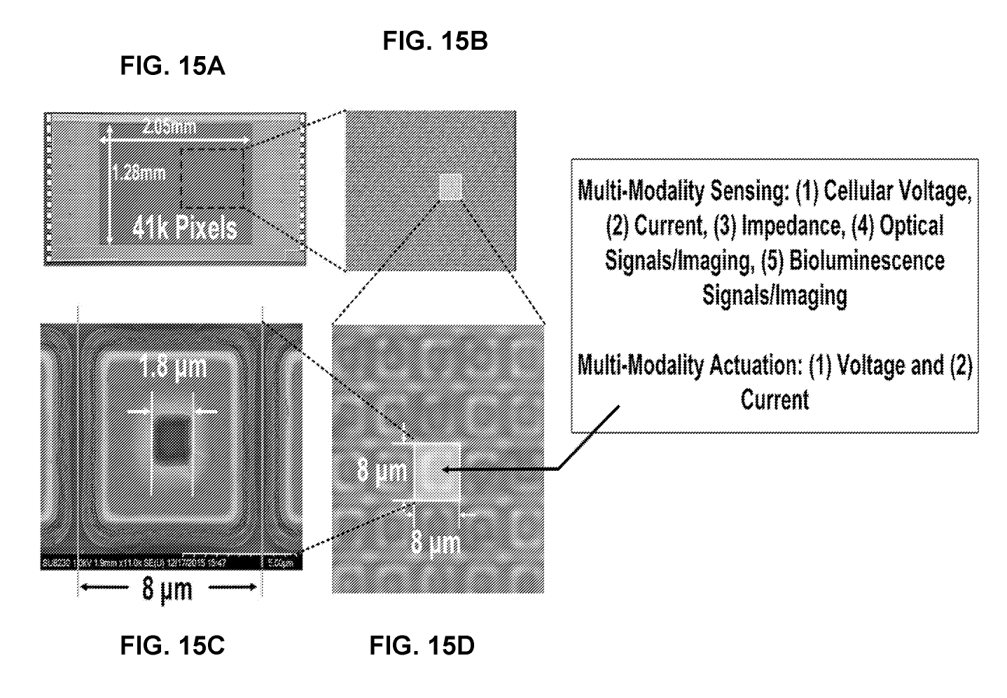

As shown in FIGS. 14 and 15, a cellular interfacing array having subcellular resolution was fabricated. FIG. 14 shows the circuit diagram and configuration of the cellular interfacing array having subcellular resolution. FIGS. 15A-15D shows micrographs demonstrating the size of each pixel of the cellular interfacing array having subcellular resolution. In this example each pixel is approximately 8 μm in length and width. Within an active cellular interfacing area of about 1.28 mm by about 2.05 mm, the cellular interfacing array had about 41,000 pixels.

This cellular interfacing array can be implemented in a standard 45 nm CMOS silicon-on-insulator (SOI) process. Each pixel is capable of performing multimodality sensing with cellular voltage, current, impedance, optical signals, and bioluminescence signals, as well as multimodality actuation with electrical voltage and current. Each pixel contains one metal pad shared by multi-modality sensing (voltage, current, impedance) and multi-modality actuation (voltage and current), a photodiode shared by multi-modality sensing (optical signals and bioluminescence signals), a few single-transistor amplifiers, and a few control switches. It does not contain any Op Amp in the pixel, and this aspect allows for the aggressive size reduction and spatial resolution improvement of this array (8 μm×8 μm/pixel).

Amplification of the sensor signals from the pixel can be provided by external amplification circuitry. As illustrated in the example of FIG. 14, pixel groups can utilize one or more Op Amp(s) that are separate from the pixels. The Op Amps can be selectively connected to pixels through a micro-switching network or multiplexer. A decoder can be used to control the switches for selection of the appropriate pixel or pixels. The output signals of the Op Amps may be conditioned (e.g., filtered), as illustrated in FIG. 14, prior to being provided for further processing or being sent to the signal processing unit (400 or 500) for cellular characterization.

In various embodiments, the multimodality sensing, signal processing, signal control, memory storage, and multimodality actuation can form a real-time or delayed close-loop feedback with either autonomous operation or non-autonomous operation controlled by external operators (either as human or machines). The real-time or delayed close-loop feedback can be used in monitoring, characterization, and regulating cells, etc. In addition, the real-time or delayed close-loop feedback can be used in monitoring, characterization, and modulating cellular, biological, chemical, and/or biochemical environments, etc. The actuation can be radio-frequency based electrical or thermal modulation or ablation, or other appropriate type of actuation. The actuators and/or sensors can be heterogeneously distributed about the array. For example, the actuators and/or sensors do not need to be in the same pixel and can be in different pixels in the same or different sub-array(s). The actuators and/or sensors can have arbitrary distributions on the cellular interfacing platform. In addition, the multimodality actuators do not have to be in the same pixel, and can have arbitrary distributions on the cellular interfacing platform. In various embodiments, multimodality actuators can be provided on the same cellular interfacing platform without the presence of sensors. Multi-modality cellular interfacing arrays (with or without actuators) with in-pixel op-amps can be implemented in CMOS nodes below 130 nm (e.g., 90 nm, 65 nm, 45 nm, etc. but excluding 130 nm). Multi-modality cellular interfacing arrays (with or without actuators) without in-pixel op-amp can be implemented in any CMOS nodes either above or below 130 nm.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A cellular interfacing array comprising:
a plurality of pixels each of which comprise:
a sensor configured to detect one or multiple types of energy from a cell, population of cells, or cellular microenvironment; and
an actuator configured to generate one or multiple types of actuation energy that is capable of actuating the cell or population of cells within responsive proximity to the actuator;
where the sensor is in direct or indirect communication with the actuator.

2. The cellular interfacing array of claim 1, wherein at least one of the pixels of the plurality of pixels comprises at least two actuators.

3. The cellular interfacing array of claim 1, wherein the cellular interfacing array is configured to actuate cells using multiple modalities.

4. The cellular interfacing array of claim 1, wherein at least one of the pixels of the plurality of pixels comprises at least two sensors.

5. The cellular interfacing array of any of claim 4, wherein the cellular interfacing array is configured to sense multiple modalities.

6. The cellular interfacing array of claim 1, wherein each pixel of the plurality of pixels comprises an operational amplifier.

7. The cellular interfacing array of claim 6, wherein the cellular interfacing array is configured using CMOS, where the CMOS is fabricated using a CMOS technology node with a minimum CMOS device gate length being greater than or equal to 130 nm.

8. The cellular interfacing array of claim 1, wherein each pixel does not contain an operational amplifier.

9. The cellular interfacing array of claim 8, wherein the cellular interfacing array is configured using CMOS, where the CMOS is fabricated using a CMOS technology node with a minimum CMOS device gate length being less than 130 nm.

10. The cellular interfacing array of claim 1, wherein the actuation energy is acoustic, optical, vibrational, thermal, electrical, magnetic, electromagnetic, radioactive, or any permissible combinations thereof.

11. The cellular interfacing array of claim 1, further comprising one or more substrates, wherein the one or more substrates can be coupled to the cellular interfacing array.

12. The cellular interfacing array of claim 11, wherein the substrate is a printed circuit board, cell culture container, biocompatible mesh, or combinations thereof.

13. The cellular interfacing array of claim 1, further comprising a transmitter and a receiver, wherein the transmitter is configured to transmit a signal from the cellular interfacing array to a processor or device having a processor, and wherein the receiver is configured to receive a signal from the processor or the device having a processor.

14. The cellular interfacing array of claim 13, wherein a transceiver comprises the transmitter and the receiver.

15. The cellular interfacing array of claim 13, wherein the device having a processor is a back-end data server.

16. The cellular interfacing array of claim 1, wherein the plurality of pixels are heterogeneous.

17. A cellular interfacing array comprising:
a plurality of pixels, where each pixel of the plurality of pixels comprises a sensor configured to detect one or multiple types of energy from a cell, population of cells, or cellular microenvironment, and
where each pixel of the plurality of pixels does not contain an operational amplifier.

18. The cellular interfacing array of claim 17, wherein each pixel of the plurality of pixels further comprises an actuator configured to generate one or multiple types of actuation energy capable of actuating the cell or population of cells within responsive proximity to the actuator, and wherein the actuator is in communication with the sensor.

19. The cellular interfacing array of claim 17, wherein at least one of the pixels of the plurality of pixels comprises at least two sensors.

20. The cellular interfacing array of claim 19, wherein the cellular interfacing array is configured to sense multiple modalities.

21. The cellular interfacing array of claim 17, wherein the cellular interfacing array is configured using CMOS, the CMOS is fabricated using a CMOS technology node with a minimum CMOS device gate length being less than 130 nm.

22. The cellular interfacing array of claim 18, wherein the one or multiple types of actuation energy is acoustic, optical, vibrational, thermal, electrical, electromagnetic, radioactive, or any combination thereof.

23. The cellular interfacing array of claim 17, further comprising one or more substrates, wherein the one or more substrates can be coupled to the cellular interfacing array.

24. The cellular interfacing array of claim 17, further comprising a transmitter and a receiver, wherein the transmitter is configured to transmit one or multiple signals from the cellular interfacing array to a device having a processor, and wherein the receiver is configured to receive one or multiple signals from the device having the processor.

25. The cellular interfacing array of claim 17, wherein the device having a processor is a remotely located cloud server.

26. The cellular interfacing array of claim 17, wherein the plurality of pixels are partially heterogeneous.

27. A medical device comprising:
a cellular interfacing array comprising:
a plurality of pixels that comprise:
a sensor configured to detect one or multiple types of energy from a cell, population of cells, or cellular microenvironment; and
an actuator configured to generate one or multiple types of actuation energy that is capable of actuating the cell or population of cells within responsive proximity to the actuator, where the sensor is in communication with the actuator.

28. A medical device comprising:
a cellular interfacing array comprising:
a plurality of pixels, where each pixel of the plurality of pixels comprises a sensor configured to detect one or multiple types of energy from a cell, population of cells, or cellular microenvironment, and
where each pixel of the plurality of pixels does not contain an operational amplifier.

29. The medical device of claim 28, wherein each pixel of the plurality of pixels further comprises an actuator configured to generate one or multiple types of actuation energy capable of actuating the cell or population of cells within responsive proximity to the actuator, and wherein the actuator is in communication with the sensor.

30. A method of detecting energy from a cell, a population of cells, or a cellular microenvironment comprising:
contacting a cell or population thereof with a cellular interfacing array, the cellular interfacing array comprising:
a plurality of pixels, where each pixel of the plurality of pixels comprises a sensor configured to detect one or multiple types of energy from a cell, population of cells, or cellular microenvironment, and
where each pixel of the plurality of pixels does not contain an operational amplifier; and
detecting the energy from the cell, population of cells, or cellular microenvironment, wherein the energy from the cell is detected by the sensor.

31. The method of claim 30, further comprising actuating the cell or the population of cells by exposing the cell or population of cells to an actuation energy generated by an actuator in a pixel of the plurality of pixels.

32. The method of claim 30, wherein at least two types of energy are detected from the cell, population of cells, or cellular microenvironment.

33. The method of claim 32, wherein the at least two types of energy are detected by the same sensor.

34. The method of claim 32, wherein the cellular interfacing array comprises at least two sensors and wherein the at least two energies are detected by different sensors.

35. The method of claim 34, wherein the different sensors are contained in the same pixel.

36. The method of claim 34, wherein the different sensors are contained in different pixels.

37. The method of claim 30, wherein the cellular interfacing array is configured using CMOS, where the CMOS is fabricated using a CMOS technology node with a minimum CMOS device gate length being greater than or equal to 130 nm.

38. The method of claim 31, wherein the actuation energy is acoustic, optical, vibrational, thermal, electrical, magnetic, electromagnetic, radioactive, or any combination thereof.

39. The method of claim 31, further comprising transmitting a first signal to a device containing a processing circuitry, wherein the device is configured to receive the first signal.

40. The method of claim 39, further comprising
receiving a second signal from the device containing processing circuitry, wherein the second signal is received by the cellular interfacing array, wherein the device is configured to transmit the second signal and wherein the receiver is configured to receive the second signal; and
actuating a cell or population of cells within responsive proximity to the actuator.

41. The method of claim 40, wherein the device containing processing circuitry provides real-time or delayed closed-loop feedback via the second signal.

42. The method of claim 30, further comprising contacting the cells with a compound of interest.

43. A method of detecting one or more types of energy from a cell, a population of cells, or a cellular microenvironment and comprising:
contacting a cell or population thereof with a cellular interfacing array, the cellular interfacing array comprising:
a plurality of pixels that comprise:
a sensor configured to detect one or more types of energy from a cell, population of cells, or cellular microenvironment; and
an actuator configured to generate one or more types of actuation energy that is capable of actuation the cell or population of cells within responsive proximity to the actuator;
where the sensor is in communication with the actuator;
detecting the energy from the cell or population thereof, where the energy is detected by the sensor; and
actuating a cell or population of cells that is in responsive proximity to the actuator by generating an actuation energy and exposing the cell or population of cells that are in responsive proximity to the actuator to the actuation energy.

44. The method of claim 43, wherein each pixel comprises an operational amplifier.

45. The method of claim 44, wherein the cellular interfacing array is configured using CMOS, where the CMOS is fabricated using a CMOS technology node with a minimum CMOS device gate length being greater than or equal to 130 nm.

46. The method of claim 43, wherein each pixel of the cellular interfacing array does not contain an operational amplifier.

47. The cellular interfacing array of claim 46, wherein the cellular interfacing array is configured using CMOS, where the CMOS is fabricated using a CMOS technology node with a minimum CMOS device gate length being less than 130 nm.

48. The method of claim 43, wherein the actuation energy is acoustic, optical, vibrational, thermal, electrical, magnetic, electromagnetic, radioactive, or any combinations thereof.

49. The method of claim 43, wherein the sensor is in communication with the actuator via a signal processing unit.

50. The method of claim 49, wherein the signal processing unit is a server.
